(19) United States
(12) Patent Application Publication
(10) Pub. No.: US 2010/0166854 A1
(43) Pub. Date: Jul. 1, 2010

(54) ELECTROSPUN MATRICES FOR DELIVERY OF HYDROPHILIC AND LIPOPHILIC COMPOUNDS

(75) Inventors: Bozena B. Michniak-Kohn, Piscataway, NJ (US); Rashmi A. Thakur, Piscataway, NJ (US); Charles A. Florek, Piscataway, NJ (US); Joachim Kohn, Piscataway, NJ (US)

Correspondence Address: FOX ROTHSCHILD LLP;PRINCETON PIKE CORPORATE CENTER, 997 LENOX DRIVE, BLDG. #3, LAWRENCEVILLE, NJ, 08648, US

(73) Assignee: Rutgers, The State University of New Jersey, New Brunswick, NJ (US)

(21) Appl. No.: 12/513,125

(22) PCT Filed: Oct. 25, 2007
(86) PCT No.: PCT/US07/82459

§ 371 Date: Mar. 22, 2010

Related U.S. Application Data
(60) Provisional application No. 60/863,517, filed on Oct. 30, 2006.

(51) Int. Cl. A61K 9/48; B29C 47/00

(52) U.S. Cl. 424/456; 264/465

(57) ABSTRACT

A method of forming electrospun fiber mats from a plurality of different biodegradable polymeric fibers is provided, in which a plurality of up to six different biodegradable polymer solutions are electrospun together by a method comprising the steps of providing a plurality of up to six different biodegradable polymer solutions each containing at least one biologically or pharmaceutically active material and each in communication with a needle for electrospinning a biodegradable polymer fiber from the solution, and pumping each solution through its respective needle into an electric field under conditions effective to produce uncontrolled charged jet streams of the polymer solutions directed at a grounded rotating mandrel, thereby forming fiber threads of the biologically or pharmaceutically active compounds and polymers in the solutions that are deposited on the mandrel to form an electrospun non-woven fiber mat, wherein the needles are positioned for co-deposition of the fiber threads from the polymer solution streams together on the mandrel to form a fiber mat.

### CROSS-REFERENCE TO RELATED APPLICATION

The present application claims priority benefit under 35 U.S.C. §119(e) of U.S. Provisional Patent Application Ser. No. 60/862,767 filed Oct. 24, 2006 and Ser. No. 60/863,517 filed Oct. 30, 2006. The disclosures of both applications are incorporated herein by reference in their entirety.

### BACKGROUND OF THE INVENTION

Nanofibers made from biocompatible and biodegradable polymers have the potential to be used for the replacement of structurally or physiologically deficient tissues and organs in humans. The use of nanofibers in tissue restoration is promising since the collagen fibers found naturally in extracellular matrix (ECM) are nano-sized objects. Cells therefore tend interact with artificial nanofibers in a way that can result in efficient, tissue restoration. Another feature of nanofibers is that they over a very large surface to volume ratio, allowing for the efficient release of pharmaceutical or biologically active agents incorporated within the nanofibers and offering large surface areas that can support cell growth. Nanofibers have been explored for wound healing; the epithelialization of implants and the construction of biocompatible prostheses, cosmetics, face masks, bone substitutes, artificial blood vessels, and valves; and drug delivery applications. Nanofibrous scaffolds designed to elicit specific cellular responses through the incorporation of signaling ligands (e.g., growth factors, adhesion peptides) or DNA fragments are viewed as particularly promising in near-term strategies. Nanoparticles and nanospheres enable controlled release of therapeutic agents, antibodies, genes, and vaccines into target cells.

Polymers such as polyglycolide (PGA), polylactide (PLA), and their random copolymer poly(glycolide-co-lactide) (PGLA) are often used as the base materials for implant devices, such as suture fibers and scaffolds, for tissue engineering. These materials meet several controlled-release criteria: they are biocompatible and biodegradable and they can provide high efficiency in drug loading. Many different techniques have been developed to produce nanostructured biodegradable materials such as microspheres, foams, and films. It has been demonstrated that the molecular structure and morphology of PLA, PGA, and their copolymers can play a major role in the degradation and mechanical properties of the final products.

Electrospinning technology is well suited to process natural biomaterials and synthetic biocompatible or bioabsorbable polymers for biomedical applications. Polycaprolactone (PCL) has been investigated mainly for long-term implants for drug release and support of mineralized tissue formation and may be a suitable substrate for the treatment of bone defects. An improvement in the mechanical properties of PCL has been achieved by copolymerization with PLA, enabling its use for orthopedic applications, such as the repair of bone defects.

Biological functioning of the organs is regulated by biologic signals from growth factors, extracellular matrix (ECM), and the surrounding cells. ECM molecules surround the cells to provide mechanical support and regulate cellular activities. The ultimate goal of the novel modified nanofibrous scaffold design is the production of an ideal structure that can replace the natural ECM until host cells can repopulate and resynthesize a new natural matrix. Collagen in its native state is a natural substrate for cell attachment, growth, and differentiation. The use of these modified nanofibers in tissue restoration is expected to result in an efficient, compact organ and a rapid recovery process owing to the large surface area offered by nanofibers made from protein used for wound healing; the epithelialization of implants and the construction of biocompatible prostheses, cosmetics, face masks, cartilage, bone substitutes, artificial blood vessels, and valves; stem cell expansion; and drug delivery applications.

Nanofibers provide a connection between the nanoscale world and the macroscale world, because the diameters can be in the nanometer range while the length of individual fibers can be in excess of many meters. Therefore, the current emphasis of research is on exploiting such properties and focusing on determining appropriate conditions for electrospinning various polymers and biopolymers for eventual applications including multi-functional membranes, biomedical structural elements (scaffolds used in tissue engineering, wound dressing, drug delivery, artificial organs, vascular grafts), protective shields in specialty fabrics, filter media for submicron particles in the separation industry, composite reinforcement, membrane filters for air purification systems, and structures for nanoelectronic machines.

Electrospinning is an atomization process of a conducting fluid that exploits the interactions between an electrostatic field and the conducting fluid. When an external electrostatic field is applied to a conducting fluid (e.g., a semi-dilute polymer solution or a polymer melt), a suspended conical droplet is formed, whereby the surface tension of the droplet is in equilibrium with the electric field. Electrostatic atomization occurs when the electrostatic field is strong enough to overcome the surface tension of the liquid. The liquid droplet then becomes unstable and a tiny jet is ejected from the surface of the droplet. As it reaches a grounded target, the material can be collected as an interconnected web containing relatively fine, i.e., small diameter, fibers. The resulting films (or membranes) from small diameter fibers have very large surface area to volume ratios and small pore sizes and are often referred to as “nanofiber mats,” “fiber mats,” “nanofibers sheets,” “fiber matrices,” “fiber meshes” or “nanofibers webs.” All of the above are used interchangeably in the literature and are understood to have the same meaning.

U.S. Pat. No. 4,323,525 is directed to a process for the production of tubular products by electrostatically spinning a liquid containing a fiber-forming material. The process introduces the liquid into an electric field through a nozzle under conditions to produce fibers of the fiber-forming material, which tend to be drawn to a charged collector, and collecting the fibers on a charged tubular collector that rotates about its longitudinal axis, to form the fibrous tubular product. It is also disclosed that several nozzles can be used to increase the rate of fiber production.

U.S. Pat. No. 4,689,186 is directed to a process for the production of polyurethane tubular products by electrostatically spinning a fiber-forming liquid containing the polyurethane. It is disclosed that auxiliary electrodes can be placed around the collector to help facilitate collection of the fibers.

U.S. Pat. No. 6,713,011 is directed to a process for electrospinning a polymer fiber from a conducting fluid containing a polymer in the presence of a first electric field modified by a second electric field to form a controlled jet stream of the conducting fluid. The second electric field can be established by imposing at least one field modifying electrode on the first electrostatic field. An embodiment is disclosed in which a plurality of spinnerets deliver different solutions with either different concentrations of polymer, different polymers, different polymer blends, different additives and/or different solvents. The controlled jet stream directs the fiber from each spinneret onto a moving support membrane directly beneath the spinneret. To the extent each spinneret delivers a different polymer, drug, or polymer-drug combination, the resulting nanofibrous sheet or web material will vary in composition in the direction trans-verse to the machine direction in which the moving support membrane travels and, in turn, the polymer degradation and drug release properties of the material will vary as well.

There remains a need for electrospun nanofibers mats suitable for in vivo implantation that are made from combinations of non-toxic and biodegradable polymers and a plurality of biologically or pharmacologically active moieties such that the polymer degradation and drug release properties can be adjusted to specific medical needs.

### SUMMARY OF THE INVENTION

This need is met by the present invention. It has now been discovered that by electrospinning onto a rotating mandrel a plurality of up to six uncontrolled jet streams of two or more different solutions, each solution containing at least one biologically or pharmaceutically active material and at least one biodegradable polymer and the two or more different solutions differing by either the concentration of the biodegradable polymer, the type of biodegradable polymer, the number of biodegradable polymers blended in the solution and/or the type or concentration of biologically or pharmaceutically active materials dissolved in the solutions, a uniformly electrospun fiber mat is formed in which an admixture of different biodegradable polymer fibers containing biologically or pharmaceutically active materials that release therefrom under physiological conditions is intermingled at the nanoscale throughout the fiber mat.

Thus, according to one aspect of the present invention, a method of forming electrospun fiber mats that appear on the macroscale to be essentially uniform in composition from a plurality of different biodegradable polymeric fibers is provided, in which a plurality of up to six different biodegradable polymer solutions are electrospun together by a method including the steps of:

providing a plurality of up to six different biodegradable polymer solutions each containing at least one biologically or pharmaceutically active material and each in communication with a needle for electrospinning a biodegradable polymer fiber from the solution; and

pumping each solution through its respective needle into an electric field under conditions effective to produce uncontrolled charged streams of polymer solution jet streams directed at a rotating mandrel of opposite charge, thereby forming fiber threads of the biologically or pharmaceutically active compounds and polymers in the solutions that are deposited on the mandrel to form an electrospun non-woven fiber mat;

wherein the needles are positioned for co-deposition of the fiber threads from the polymer solution streams together on the mandrel to form a fiber mat that appears to be essentially uniform in composition when observed on the macroscale, but without merging any two or more polymer streams into a single electrospun fiber.

For purposes of the present invention, the terms “non-woven fiber mat,” “nanofiber mats,” “fiber mats,” “nanofiber sheets,” “fiber matrices,” “fiber meshes” and “nanofibers webs” are used interchangeably.

According to one embodiment of the present invention two or more solutions each contain a different biodegradable polymer. According to another embodiment of the present invention, at least two solutions contain the same biodegradable polymer, but at different solution concentrations. According to yet another embodiment of the invention, at least one solution contains two or more biodegradable polymers.

According to one embodiment of the present invention, two or more solutions each contain a different biologically active or pharmaceutically active material. According to another embodiment of the invention, at least two solutions contain the same biologically or pharmaceutically active material, but at different solution concentrations. According to yet another embodiment of the invention, at least one solution contains two or more biologically or pharmaceutically active materials. According to yet another embodiment at least one solution contains an extracellular matrix protein, for example collagen, laminin, fibronectin, vitronectin, or a combination thereof, which is then incorporated into a fiber. Yet another embodiment contains a peptide, a cytokine, or a cell signaling molecule, or a combination thereof, which is then incorporated into a fiber.

According to one embodiment of the invention, a first solution contains a first biodegradable polymer and a first biologically or pharmaceutically active material and a second solution contains a second biodegradable polymer and a second biologically or pharmaceutically active material. According to another embodiment of the invention, the first biologically or pharmaceutically active material is compatible with the first biodegradable polymer but incompatible with the second biodegradable polymer, or the second biologically or pharmaceutically active material is compatible with the second biodegradable polymer but incompatible with the first biodegradable polymer, or both. According to yet another embodiment of the invention, the first and second biologically or pharmaceutically active material are incompatible with each other.

According to an embodiment of the invention, two or more solutions contain the same biodegradable polymer and biologically or pharmaceutically active material but different solvents. According to another embodiment of the invention, the biologically or pharmaceutically active material is not released from the biodegradable polymer matrix. According to yet another embodiment, the biologically or pharmaceutically active material that is not released, but is expressed at the fiber surface and interacts with the environment.

The inventive method provides polymer fiber mats containing two or more different biodegradable polymer fibers, or two or more different biologically or pharmaceutically active materials released from the same or different biodegradable polymer fibers, or both. Therefore, according to another aspect of the present invention, biodegradable polymer fiber mats suitable for in vivo implantation are provided that are prepared by the electrospinning method according to the method of the present invention.

According to one embodiment of the invention the polymer fiber mats contain at least one fiber less than about 100 microns in diameter. According to another embodiment of the invention, the polymer fiber mats contain at least one fiber less than about 10 microns in diameter. According to further embodiments of the invention polymer fiber mats are provided according to the foregoing embodiments in which essentially all the fiber diameters do not exceed the defined maximum diameter.

According to one embodiment of the invention, the polymer fiber mats contain at least one fiber less than 1 micron in diameter. According to one embodiment of the invention the polymer fiber mats contain at least one fiber less than about 500 nanometers in diameter. According to another embodiment of the invention, the polymer fiber mats contain at least one fiber less than about 100 nanometers in diameter. According to another embodiment of the invention, the polymer fiber mats contain at least one fiber less than about 10 nanometers in diameter. According to further embodiments of the invention polymer fiber mats are provided according to the foregoing embodiments in which essentially all the fiber diameters do not exceed the defined maximum diameter.

The biologically and pharmaceutically active materials, the biodegradable polymers, and the level of loading of the biologically and pharmaceutically active materials can be selected to provide a polymer matrix with a predetermined release profile. The release profile can include an essentially sustained release, an essentially sustained release following an initial lag or an initial burst, essentially an entirely single burst release, either immediately or after an initial lag, or an alternating series of plural bursts and lags following an initial burst or lag.

The biodegradable polymer matrices according to the present invention have utility as implantable medical devices such as barriers for the prevention of surgical adhesions, wound dressings, drug delivery devices, including capsules for oral or rectal administration, subcutaneous implants, transdermal drug delivery devices and other occlusive and non-occlusive skin and buccal patches, polymer scaffolds for tissue engineering, and the like. Oral dosage forms include rolled up fiber mats placed into gelatin capsules for oral administration.

The foregoing and other objects, features and advantages of the present invention are more readily apparent from the detailed description of the preferred embodiments set forth below, taken in conjunction with the accompanying drawings.

### BRIEF DESCRIPTION OF THE DRAWINGS

FIG. 1 depicts a schematic of a composite, drug delivery fiber mat wherein or illustration purposes, Drug A containing fibers have been pseudocolored white and Drug B containing fibers have been pseudocolored dark;

FIG. 2 depicts the release from three separate formulations of a peptide drug over time as a function of the molecular weight of a polymeric excipient;

FIG. 3 depicts a dual needle electrospinning apparatus according to the present invention;

FIG. 4 depicts logarithmically the distribution of fiber diameters in a polymeric mesh electrospun according to the double needle (DS) method of the present invention in comparison to the distribution of fiber diameters in a polymeric mesh electrospun according to the single needle (SS) method of the prior art;

FIG. 5 depicts the release profiles of (a) lidocaine hydrochloride and (b) mupirocin incorporated in PLLA and electrospun by the double needle method according to the present invention and the single needle prior art technique;

FIG. 6 depicts from bottom to top DSC thermograms of mupirocin only, mupirocin electrospun by the double needle method according to the present invention, and mupirocin and lidocaine hydrochloride electrospun by the single needle method of the prior art, in which crystallization of the lidocaine hydrochloride and mupirocin from the polymer domains is indicated; and

FIG. 7 depicts mupirocin release in a Franz cell receptor from a dual fiber polymeric matrix according to the present invention compared to the MIC for mupirocin.

### DETAILED DESCRIPTION OF THE INVENTION

The present invention relates to electrospinning a fiber from a polymer solution of at least one polymer and at least one drug to form an electrospun polymeric scaffold containing one or more drugs for delivery therefrom. Electrospinning involves producing fibers with the help of an electrical field. Solutions of solids when passed between charged electrodes separate into threads which are then collected on a charged collector. Electrospinning is capable of producing fiber diameters in the nanometer to micrometer range.

The basic setup of an electrospinning apparatus according to the present invention includes a high voltage power supply, a plurality of electrospinning needles, and a grounded collector, here a rotating mandrel. With the aid of a syringe connected to a pump, polymer solutions can be fed at a controlled rate through the needles. Under high voltage, the drops at the tip of the needles become electrified and uniformly charged all over their surfaces. The electrostatic repulsion between the surface charges and the coulombic force exerted by the external electric field force the drops into the form of Taylor cones.

With increasing strength of the applied electric field, the electrostatic forces overcome the surface tension in the polymer drop and force jets out of the needles which in an attempt to reach the grounded collector whip into sprays. The optimal tip-to-collector distance and the high surface area of the fibers assist in complete evaporation of the solvent from the fibers before reaching the collector. While conventionally it has been believed that the jets form the Taylor cone by dividing into a number of small splayed fibers, each jet is actually a single rapidly-rotating spiral fiber in a whipping motion which gives an illusion of a cone, referred to as a Taylor cone.

The needles are positioned for co-deposition of fiber threads from the polymer solution jet streams together on the rotating mandrel to form a fiber mat essentially uniform in composition at the macroscale, but consisting of individually distinct fibers on the nanoscale. When more than two needles are employed they are arrayed over the rotating mandrel in a non-linear fashion, for example, the needles can be positioned to define the corners of a polygon or the circumference of a circle. In one configuration employing “n” number of needles, n−1 needles define the corners of a polygon or the circumference of a circle, with the nth needle in the center.

In particular, the process of electrospinning involves the use of a polymer solution which is placed in a syringe. A controllable pump ejects the solution from the syringe needle at a predetermined rate. The surface tension holds the solution at the tip of the needle together in the form of a droplet. An external electric field is induced and as is the field strength increased, the charges created directly oppose the surface-interfacial tension force. At a critical value these forces cause the ejection of a jet stream from the droplet and the formation of the Taylor cone at the end of the needle which was described above. During its spiral path, the solution evaporates and the jet stream begins to thin, leaving behind a polymer fiber that is collected on a grounded electrically conducting surface. Continuous fibers are laid on the top of the conducting surface and finally form a non woven fiber mat.

In the case of this invention, the electrically conducting surface is part of a rotating mandrel so that after each 360 degree turn of the mandrel, the same area is exposed to the descending jet streams, allowing multiple layers of electrospun fibers to be deposited on top of each other till the resulting fiber mat has the desired thickness. In addition to its rotating motion, the collecting mandrel can also be moved along its long axis back and forth. In this way, a mandrel that is longer than the collection area of the Taylor cone can be used and uniformly covered with a fiber mat of desired thickness.

When the solutions are delivered simultaneously, a single layer mixed fiber fabric is produced. When the solutions are delivered sequentially, each needle produces one layer of fibers, which results in a multilayered fiber fabric.

This invention addresses several limitations and needs of current drug delivery technologies:

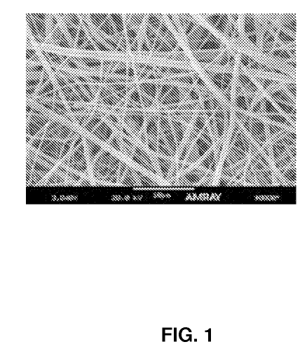

In the first scenario, a first drug, referred to as “Drug A” and a second drug, referred to as “Drug B” have a synergistic, beneficial effect on the patient and should, for best patient benefit, be co-delivered to the same site within the body of the patient but require different release profiles. In this case, it is not generally possible to formulate a single polymeric release device that can provide optimum release profiles for each of the drugs. By formulating Drug A within one type of electrospun fiber, and Drug B in a differently formulated electrospun fiber, it is possible to optimize each polymeric drug delivery fiber type with respect to the required drug release rate. By co-spinning the two different formulations and co-depositing the resulting fibers as in intimate and intertwined mixture of tiny fibers within the same fiber mat, the objective of effective co-delivery of two different drugs, each having its own optimized drug release profile, can be realized using one single delivery device as illustrated in FIG. 1.

This invention also addresses the problem presented when Drug A and Drug B are physically incompatible and cannot be formulated within the same device. For example, any drug combination where one drug is an oxidizer and the other drug is a reducing agent, or one drug is an acid while the other is a base, may lead to compatibility and drug stability (shelf life) problems when such drugs are co-formulated within the same polymeric matrix.

This invention further addresses the problem of simultaneous delivery of multiple peptides, proteins, or oligonucleotides. Electrospinning is known in the art to be a mild fabrication method that is useful for the formulation of sensitive biological agents such as peptides, proteins or oligonucleotides within polymeric matrices. It is expected that peptide and protein drugs (including vaccines, cytokines and cell signaling molecules) will be more widely used as therapeutic agents in the future.

This invention also addresses the problem of pulsatile release. As illustrated in US Patent Application Publication No. 2003-0216307, the disclosure of which is incorporated herein by reference in its entirety, polymeric drug formulations can be prepared that release an embedded drug in a burst like fashion after a pre-programmed delay.

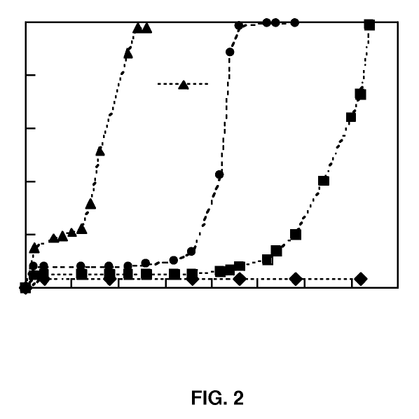

U.S. Patent Application Publication No. 2003-0216307 teaches the preparation of individual release formulations each providing a burst-like release after a given delay time. This is illustrated in FIG. 2 showing the release from three separate formulations of a peptide drug (Integrilin) over time as a function of the molecular weight of a polymeric excipient. Within the context of this invention, a plurality of such individual formulations could be combined as individual fiber components within a single fiber mat. In the example provided here, the resulting fiber mat, after implantation in the body of a patient would release a burst of drug 6 days, 18 days and about 30 days after implantation of the drug release device.

This type of “burst like” pulsatile release is particularly useful in single step immunization protocols that require multiple administration of the same antigen. A burst release of a drug is possible when the drug is more lipophilic (e.g. hydrophobic) or less lipophilic (e.g., hydrophilic) compared to polymer of the fiber into which the drug is incorporated. A sustained release of a drug is possible when the lipophilicity of the drug is similar to that of the polymer in the fiber.

The fiber matrices are envisioned to be implantable devices (for example for prevention of surgical adhesions or for single step immunization or contraception protocols). They can also be formulated to be inserted to fill tissue defects in wound care and wound healing applications. They can also be formulated as wound dressings, including wound dressings containing antibiotics that prevent or treat methicillin resistant Staphylococcus aureus (MRSA) infections. A fourth area of utility of such fiber mats is in personalized medicine where the drug loaded fiber mat is presented within a standard oral capsule for the convenient, oral administration of combinations of drugs that cannot otherwise be prepared within a single formulation.

One wound dressing embodiment of this invention is when the drug containing fiber mat is embedded within a conventional wound dressing hydrogel. The incorporation of a thin nylon mesh (for better handling properties) and the addition of some moisture control backing are optional features of wound dressings that can be readily implemented as needed. Optionally, an extracellular matrix protein, for example collagen, laminin, fibronectin, vitronectin, or a combination thereof, is incorporated into a fiber.

A fifth area of utility is in hormone delivery. The release profile can also be formulated using estrogens and/or progestogens to modify the menstrual cycle for purposes of contraception, to modulate excessive variations in hormone levels or to replace hormones no longer produces following menopause. The fiber mat can be administered for extended hormone delivery.

The present invention can also be used in cosmetic applications to deliver one or more active agents for an extended period of time, preferably overnight. Preferred active agents for cosmetic applications include those typically used in the cosmetic arts.

In another embodiment, fiber mat is secured by tape or an adhesive layer to the area to be treated. The adhesive layer would either cover the entire surface of the mat or be coated on the periphery of the area to make skin contact, or both. The surface of the fabric facing away from the skin can include an adhesive laminated or heat-bonded to a protective backing that is either occlusive or air-permeable.

Transdermal drug delivery devices can be fabricated by the lamination of an occlusive backing to a fiber mat. When an occlusive backing is used with a larger surface area than the fiber mat, the excess surface area can be coated with an adhesive suitable for skin contact for affixing the patch to the skin of the patient. According to one embodiment at least one fiber is loaded with a biologically or pharmaceutically active agent and at least one fiber is loaded with a penetration enhancer. According to another embodiment, at least one fiber is loaded with an anti-inflammatory agent to relieve the inflammation that often accompanies transdermal drug delivery. A contraceptive patch can be prepared using the above-described fiber matrices loaded with estrogens and/or progestogens.

Any biocompatible electrospinnable polymer is suitable for use in the present invention. Electrospinnable polymers include those that are soluble in at least one organic solvent or water and have sufficiently high molecular weight to be above the “chain entanglement point,” which is defined as the minimum molecular weight needed for the polymer to form a self-supporting film by solvent casting. One of skill in the art is capable of determining the chain entanglement point of a polymer. The polymer can be biodegradable or non-biodegradable. In one embodiment, the wound dressing is inserted into a wound of a patient. Preferred patients include mammals, for example, humans, horses, pigs, cattle, dogs, and cats.

Suitable polymers include polysaccharides, poly(alkylene oxides), polyarylates, for example those disclosed in U.S. Pat. No. 5,216,115, block co-polymers of poly(alkylene oxides) with polycarbonates and polyarylates, for example those disclosed in U.S. Pat. No. 5,658,995, polycarbonates and polyarylates, for example those disclosed in U.S. Pat. No. 5,670,602, free acid polycarbonates and polyarylates, for example those disclosed in U.S. Pat. No. 6,120,491, polyamide carbonates and polyester amides of hydroxy acids, for example those disclosed in U.S. Pat. No. 6,284,862, polymers of L-tyrosine derived diphenol compounds, including polythiocarbonates and polyethers, for example those disclosed in U.S. Pat. No. RE37,795, strictly alternating poly(alkylene oxide) ethers, for example those disclosed in U.S. Pat. No. 6,602,497, polymers listed on the United States FDA “EAFUS” list, including polyacrylamide, polyacrylamide resin, modified poly(acrylic acid-co-hypophosphite), sodium salt polyacrylic acid, sodium salt poly(alkyl(C16-22) acrylate), polydextrose, poly(divinylbenzene-co-ethylstyrene), poly(divinylbenzene-co-trimethyl(vinylbenzyl)ammonium chloride), polyethylene (m.w. 2,00-21,000), polyethylene glycol, polyethylene glycol (400) dioleate, polyethylene (oxidized), polyethyleneimine reaction product with 1,2-dichloroethane, polyglycerol esters of fatty acids, polyglyceryl phthalate ester of coconut oil fatty acids, polyisobutylene (min. m.w. 37,000), polylimonene, polymaleic acid, polymaleic acid, sodium salt, poly(maleic anhydride), sodium salt, polyoxyethylene dioleate, polyoxyethylene (600) dioleate, polyoxyethylene (600) mono-rici noleate, polyoxyethylene 40 monostearate, polypropylene glycol (m.w. 1,200-3,000), polysorbate 20, polysorbate 60, polysorbate 65, polysorbate 80, polystyrene, cross-linked, chloromethylated, then aminated with trimethylamine, dimethylamine, diethylenetriamine, or triethanolamine, polyvinyl acetate, polyvinyl alcohol, polyvinyl pyrrolidone, and polyvinylpyrrolidone, and polymers listed in U.S. Pat. No. 7,112,417, the disclosures of all of which are incorporated herein by reference in their entirety.

Single step immunization protocols administer one or more doses of one or more vaccine agents and optionally co-deliver one or more adjuvants. Vaccines function by triggering the immune system to mount a response to an agent, or antigen. Typically the vaccine is in the form of an infectious organism or a portion thereof that is introduced into the body in a non-infectious or non-pathogenic form. Once the immune system has been “primed” or sensitized to the organism, later exposure of the immune system to this organism as an infectious pathogen results in a rapid and robust immune response that destroys the pathogen before it can multiply and infect enough cells in the host organism to cause disease symptoms.

The agent, or antigen, used to prime the immune system can be the entire organism in a less infectious state, known as an attenuated organism, or in some cases, components of the organism such as carbohydrates, proteins or peptides representing various structural components of the organism.

The present invention therefore includes fiber matrices for delivery of a vaccine in which at least one fiber contains a vaccine agent. The vaccine agents include vaccines and antigens derived from infectious viruses of both human and non-human vertebrates, include retroviruses, RNA viruses and DNA viruses. This group of retroviruses includes both simple retroviruses and complex retroviruses. The simple retroviruses include the subgroups of B-type retroviruses, C-type retroviruses and D-type retroviruses. An example of a B-type retrovirus is mouse mammary tumor virus (MMTV). The C-type retroviruses include subgroups C-type group A (including Rous sarcoma virus (RSV), avian leukemia virus (ALV), and avian myeloblastosis virus (AMV)) and C-type group B (including murine leukemia virus (MLV), feline leukemia virus (FeLV), murine sarcoma virus (MSV), gibbon ape leukemia virus (GALV), spleen necrosis virus (SNV), reticuloendotheliosis virus (RV) and simian sarcoma virus (SSV)). The D-type retroviruses include Mason-Pfizer monkey virus (MPMV) and simian retrovirus type 1 (SRV-1). The complex retroviruses include the subgroups of lentiviruses, T-cell leukemia viruses and the foamy viruses. Lentiviruses include HIV-1, but also include HIV-2, SIV, Visna virus, feline immunodeficiency virus (FIV), and equine infectious anemia virus (EIAV). The T-cell leukemia viruses include HTLV-1, HTLV-II, simian T-cell leukemia virus (STLV), and bovine leukemia virus (BLV). The foamy viruses include human foamy virus (HFV), simian foamy virus (SFV) and bovine foamy virus (BFV).

Examples of other RNA viruses that are antigens in mammals include, but are not limited to, the following: members of the family Reoviridae, including the genus Orthoreovirus (multiple serotypes of both mammalian and avian retroviruses), the genus Orbivirus (Bluetongue virus, Eugenangee virus, Kemerovo virus, African horse sickness virus, and Colorado Tick Fever virus), the genus Rotavirus (human rotavirus, Nebraska calf diarrhea virus, murine rotavirus, simian rotavirus, bovine or ovine rotavirus, avian rotavirus); the family Picornaviridae, including the genus Enterovirus (poliovirus, Coxsackie virus A and B, enteric cytopathic human orphan (ECHO) viruses, hepatitis A virus, Simian enteroviruses, Murine encephalomyelitis (ME) viruses, Poliovirus muris, Bovine enteroviruses, Porcine enteroviruses, the genus Cardiovirus (Encephalomyocarditis virus (EMC), Mengovirus), the genus Rhinovirus (Human rhinoviruses including at least 113 subtypes; other rhinoviruses), the genus Apthovirus (Foot and Mouth disease (FMDV); the family Calciviridae, including Vesicular exanthema of swine virus, San Miguel sea lion virus, Feline picornavirus and Norwalk virus; the family Togaviridae, including the genus Alphavirus (Eastern equine encephalitis virus, Semliki forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes).

Examples of other RNA viruses also include Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); the family Rhabdoviridae, including the genus Vesiculovirus (VSV), Chandipura virus, Flanders-Hart Park virus), the genus Lyssavirus (Rabies virus), fish Rhabdoviruses, and two probable Rhabdoviruses (Marburg virus and Ebola virus); the family Arenaviridae, including Lymphocytic choriomeningitis virus (LCM), Tacaribe virus complex, and Lassa virus; the family Coronoaviridae, including Infectious Bronchitis Virus (IBV), Mouse Hepatitis virus, Human enteric corona virus, and Feline infectious peritonitis (Feline coronavirus).

Illustrative DNA viruses that are antigens in mammals include, but are not limited to: the family Poxyiridae, including the genus Orthopoxvirus (Variola major, Variola minor, Monkey pox Vaccinia, Cowpox, Buffalopox, Rabbitpox, Ectromelia), the genus Leporipoxvirus (Myxoma, Fibroma), the genus Avipoxvirus (Fowlpox, other avian poxvirus), the genus Capripoxvirus (sheeppox, goatpox), the genus Suipoxvirus (Swinepox), the genus Parapoxvirus (contagious postular dermatitis virus, pseudocowpox, bovine papular stomatitis virus); the family Iridoviridae (African swine fever virus, Frog viruses 2 and 3, Lymphocystis virus of fish); the family Herpesviridae, including the alpha-Herpesviruses (Herpes Simplex Types 1 and 2, Varicella-Zoster, Equine abortion virus, Equine herpes virus 2 and 3, pseudorabies virus, infectious bovine keratoconjunctivitis virus, infectious bovine rhinotracheitis virus, feline rhinotracheitis virus, infectious laryngotracheitis virus) the Beta-herpesvirises (Human cytomegalovirus and cytomegaloviruses of swine, monkeys and rodents); the gamma-herpesviruses (Epstein-Barr virus (EBV), Marek's disease virus, Herpes saimiri, Herpesvirus ateles, Herpesvirus sylvilagus, guinea pig herpes virus, Lucke tumor virus); the family Adenoviridae, including the genus Mastadenovirus (Human subgroups A, B, C, D, E and ungrouped; simian adenoviruses (at least 23 serotypes), infectious canine hepatitis, and adenoviruses of cattle, pigs, sheep, frogs and many other species, the genus Aviadenovirus (Avian adenoviruses); and non-cultivatable adenoviruses; the family Papoviridae, including the genus Papillomavirus (Human papilloma viruses, bovine papilloma viruses, Shope rabbit papilloma virus, and various pathogenic papilloma viruses of other species), the genus Polyomavirus (polyomavirus, Simian vacuolating agent (SV-40), Rabbit vacuolating agent (RKV), K virus, BK virus, JC virus, and other primate polyoma viruses such as Lymphotrophic papilloma virus); the family Parvoviridae including the genus Adeno-associated viruses, the genus Parvovirus (Feline panleukopenia virus, bovine parvovirus, canine parvovirus, Aleutian mink disease virus, etc). Finally, DNA viruses may include viruses which do not fit into the above families such as Kuru and Creutzfeldt-Jacob disease viruses and chronic infectious neuropathic agents (CHINA virus).

Specific examples of HIV antigens can be, without any limitation, one or several antigens derived from Tat, gp120, gp160, gag, pol, protease, and nef. Other exemplary antigens are HPV antigens from any strain of HPV and antigens obtained or derived from the hepatitis family of viruses, including hepatitis A virus (HAV), hepatitis B virus (BBV), hepatitis C virus (HCV), the delta hepatitis virus (HDV), hepatitis E virus (BEV) and hepatitis G virus (HGV). See, e.g., International Publication Nos. WO 89/04669; WO 90/11089; and WO 90/14436.

In like manner, a wide variety of proteins from the herpesvirus family can be used as antigens in the present invention, including proteins derived from herpes simplex virus (HSV) types 1 and 2, such as HSV-I and HSV-2 glycoproteins gB, gD and gH; antigens from varicella zoster virus (VZV), Epstein-Barr virus (EBV) and cytomegalovirus (CMV) including CMV gB, and gH; and antigens from other human herpesviruses such as HHV6 and HAV7.

Antigens or vaccines may also be derived from respiratory syncytial virus (RSV), a negative strand virus of the paramyxoviridae family and a major cause of lower pulmonary tract disease, particularly in young children and infants.

Other vaccine agents which can be used include Influenza Virus Vaccines. Recombinant cold-adapted/temperature-sensitive influenza virus strains that can be used as vaccines have a viral coat presenting influenza virus hemagglutinin (HA) and neuraminidase (NA) immunogenic epitopes from a virulent influenza strain along with an attenuated influenza virus core.

Vaccine agents also include vaccines and antigens may be derived from bacteria, parasites or yeast. Examples of suitable species include Neisseria spp, including N. gonorrhea and N. meningitidis (including capsular polysaccharides and conjugates thereof, transferrin-binding proteins, lactoferrin binding proteins, PilC and adhesions); S. pyogenes (including M proteins or fragments thereof, C5A protease, lipoteichoic acids), S. agalactiae, S. mutans; H. ducreyi; Moraxella spp, including M. catarrhalis, also known as Branhamella catarrhalis (including high and low molecular weight adhesins and invasins); Bordetella spp, including B. pertussis (including pertactin, pertussis toxin or derivatives thereof, filamenteous hemagglutinin, adenylate cyclase, fimbriae), B. parapertussis and B. bronchiseptica. Examples of other suitable species include Mycobacterium spp., including M. tubercolosis (including ESAT6, Antigen 85A, -B or -Q, M. bovis, M leprae, M. avium, M. paratuberculosis, M. smegmatis; Legionella spp, including L. pneumophila; Escherichia spp, including enterotoxic E. coli (including colonization factors, heat-labile toxin or derivatives thereof, heat-stable toxin or derivatives thereof), enterohemorragic E. coli, enteropathogenic E. coli (including Vibrio shiga toxin-like toxin or derivatives thereof); Vibrio spp, including V. cholera (for example cholera toxin or derivatives thereof; Shigella spp, including S. sonnei, S. dysenteriae, S. flexnerii; Yersinia spp, including Y. enterocolitica (including a Yop protein), Y. pestis, Y. pseudotuberculosis; Campylobacter spp, including C. jejuni (including toxins, adhesins and invasins) and C coli; Salmonella spp, including S. typhip, S. paratyphi, S. choleraesuis, S. enteritidis; Listeria spp., including L. monocytogenes; Helicobacter spp, including H. pylori (including urease, catalase, vacuolating toxin).

Examples of other suitable bacteria species include Pseudomonas spp, including P. aeruginosa; Staphylococcus spp., including S. aureus, S. epidermidis; Enterococcus spp., including E. jaecalis, E. jaecium; Clostridium spp., including C. tetani (including tetanus toxin and derivatives thereof), C. botulinum (including botulinum toxin and derivatives thereof, C. difficile (including clostridium toxins A or B and derivatives thereof); Bacillus spp., including B. anthracis (including botulinum toxin and derivatives thereof); Corynebacterium spp., including C. diphtheriae (including diphtheria toxin and derivatives thereof); Borrelia spp., including B. burgdorferi (including OspA, OspC, DbpA, DbpB), B. garinii (including OspA, OspC, DbpA, DbpB), B. afzelii (including OspA, OspC, DbpA, DbpB), B. andersonii (including OspA, OspC, DbpA, DbpB), B. hermsii; Ehrlichia spp., including E. equi and the agent of the Human Granulocytic Ehrlichiosis; Rickettsia spp, including R. rickettsii; Chlamydia spp., including C. trachomatis (including MOMP, heparin-binding proteins), C. pneumoniae (including MONT, heparin-binding proteins), C. psittaci; Leptospira spp., including L. interrogans; Treponema spp., including T. pallidum (including the rare outer membrane proteins), T. denticola, T. hyodysenteriae; or species derived from parasites such as Plasmodium spp., including P. falciparum; Toxoplasma spp., including T. gondii (including SAG2, SAG3, Yg34); Entamoeba spp., including E. histolytica; Babesia spp., including B. microti; Trypanosoma spp., including T. cruzi; Giardia spp., including G. lamblia; Leshmania spp., including L. major; Pneumocystis spp., including P. carinii; Trichomonas spp., including T. vaginalis; Schisostoma spp., including S. mansoni, or species derived from yeast such as Candida spp., including C albicans; Cryptococcus spp., including C neoformans.

Vaccine agents also include cancer antigens and tumor antigens, including compounds, such as peptides, associated with a tumor or cancer cell surfaces that are capable of provoking an immune response when expressed on the surface of an antigen presenting cell in the context of an MHC molecule. Cancer antigens can be prepared from cancer cells either by preparing crude extracts of cancer cells, for example, as described in Cohen, et al., Cancer Research, 54, 1055 (1994), by partially purifying the antigens, by recombinant technology, or by de novo synthesis of known antigens. Cancer antigens include antigens that are recombinantly an immunogenic portion of or a whole tumor or cancer. Such antigens can be isolated or prepared recombinantly or by any other means known in the art.

Tumor antigens useful for the immunotherapeutic treatment of cancers include tumor rejection antigens such as those for prostate, breast, colorectal, lung, pancreatic, renal, ovarian or melanoma cancers. Exemplary antigens include MAGE 1 and MAGE 3 or other MAGE antigens (for the treatment of melanoma), and PRAME, BAGE, or GAGE antigens. Suitable antigens are expressed in a wide range of tumor types, such as melanoma, lung carcinoma, sarcoma and bladder carcinoma. Other tumour-specific antigens include, but are not restricted to, tumour-specific gangliosides, Prostate specific antigen (PSA) or Her-2/neu, KSA (GA733), PAP, manunaglobin, MUC-1, carcinoembryonic antigen (CEA).

Tumor antigens also include antigens associated with tumor-support mechanisms (e.g. angiogenesis, tumor invasion). Additionally, antigens particularly relevant for vaccines in the therapy of cancer also comprise Prostate-specific membrane antigen (PSMA), Prostate Stem Cell Antigen (PSCA), tyrosinase, survivin, NY-ES01, prostase, PS108 (WO 98/50567), RAGE, LAGE, HAGE.

Vaccine agents also include agents for the prophylaxis or therapy of allergy. Such vaccines would comprise allergen specific (for example Der p 1) and allergen non-specific antigens (for example peptides derived from human IgE, including but not restricted to the stanworth decapeptide (EP 0 477 231 B1)).

Vaccines agents also include antigens for the prophylaxis or therapy of chronic disorders such as atherosclerosis, and Alzheimer's disease. Antigens relevant for the prophylaxis and the therapy of patients susceptible to or suffering from Alzheimer neurodegenerative disease are, in particular, the N terminal 39-43 amino acid fragment (AP) of the amyloid precursor protein and smaller fragments (WO 99/27944).

In many cases, it is necessary to enhance the immune response to the antigens present in a vaccine in order to stimulate the immune system to a sufficient extent to make a vaccine effective, i.e., to confer immunity. To this end, additives (adjuvants) have been devised which immobilize antigens and stimulate the immune response. Mechanisms of adjuvant action are reviewed in PCT publication no. WO 03/009812. The present invention therefore includes fiber matrices for delivery of a vaccine in which at least one fiber contains a vaccine adjuvant.

Examples of adjuvants include, but are not limited to, oil-emulsion and emulsifier-based adjuvants such as complete Freund's adjuvant, incomplete Freund's adjuvant, MF59, or SAF; mineral gels such as aluminum hydroxide (alum), aluminum phosphate or calcium phosphate; microbially-derived adjuvants such as cholera toxin (CT), pertussis toxin, Escherichia coli heat-labile toxin (LT), mutant toxins (e.g., LTK63 or LTR72), Bacille Calmette-Guerin (BCG), Corynebacterium parvum, DNA CpG motifs, muramyl dipeptide, or monophosphoryl lipid A; particulate adjuvants such as immunostimulatory complexes (ISCOMs), liposomes, biodegradable microspheres, or saponins (e.g., QS-21); synthetic adjuvants such as nonionic block copolymers, muramyl peptide analogues (e.g., N-acetyl-muramyl-L-threonyl-D-isoglutamine [thr-MDP], N-acetyl-nor-muramyl-L-alanyl-D-isoglutamine, N-acetylmuramyl-L-alanyl-D-isoglutaminyl-L-alanine-2-[1′-2′-dipalmitoyl-s-n-glycero-3-hydroxy-phospho-ryloxy]-ethylamine), polyphosphazenes, or synthetic polynucleotides, and surface active substances such as lysolecithin, pluronic polyols, polyanions, peptides, hydrocarbon emulsions, or keyhole limpet hemocyanins (KLH). Other adjuvants include cytokines. Non-limiting examples of cytokines, which may be used alone or in combination include, interleukin-2 (IL-2), stem cell factor (SCF), interleulin 3 (IL-3), interleukin 6 (IL-6), interleukin 12 (IL-12), G-CSF, granulocyte macrophage-colony stimulating factor (GM-CSF), interleukin-1 alpha (IL-1.alpha.), interleukin-11 (IL-11), MIP-1.alpha., leukemia inhibitory factor (LIP), c-kit, ligand, thrombo-poietin (TPO), CD40 ligand (CD40L), tumor necrosis factor-related activation-induced cytokine (TRANCE) and flt3 ligand (flt-3L). Cytokines are commercially available from several vendors such as, for example, Genzyme, Genentech, Amgen and Immunex. Preferably, these additional adjuvants are also pharmaceutically acceptable for use in humans.

Polymer matrices according to the present invention can also be fabricated to prevent postoperative adhesions (POA). Adhesion formation is a complication of wound healing after surgery, especially abdominal surgery, that is a significant cause of post-operative morbidity. The cellular events in wound healing are mediated by an array of cytokines functioning as chemoattractants and immunostimulants. Their role in adhesion formation has become increasingly apparent in recent years. Adhesiogenic cytokines have included interleukin-6 and interleukin-1a, transforming growth factor-α, and transforming growth factor-β, epidermal growth factor, and tumor necrosis factor-α. Interleukin-10 has been shown to reduce adhesion formation by inhibiting the formation of IL-1, IL-6, and TNF-α. Various non-steroidal anti-inflammatory agents have been shown to reduce adhesion formation. Thus, the use of agents that inhibit the inflammatory cascade may have a unique role in minimizing adhesion formation.

The present invention therefore includes fiber matrices for preventing adhesion formation in which at least one fiber contains a bioactive agent for preventing surgical adhesions. Among the useful bioactive agents for preventing surgical adhesions are peptides, including LHRH (e.g., tryptoroline), somatostatin analogs (e.g., lanreotide and octreotide), and bombesin. Another group of bioactive agents includes (1) potent, non-steroidal anti-inflammatory drugs (e.g., naproxen, Tolmetin); (2) anti-neoplastic/anti-proliferative drugs (e.g., paclitaxel); (3) drugs which exhibit more than one mode of pharmacological activity, such as trapidil, which is an anti-inflammatory drug that inhibits cell aggregation; and (4) interleukin-4 (IL-4). Another bioactive agent is an ionic conjugate of two different bioactive molecules with different mechanisms of action, but can synergistically prevent POA. Typical examples of these ionic conjugates are those comprising (1) a basic peptide (e.g., lanreotide) and an acidic NSAID, such as naproxen; and (2) low molecular weight heparin and a basic peptide.

Exemplary bioactive agents which may be delivered include, for example, anticoagulants, for example heparin and chondroitin sulfate, fibrinolytics such as tPA, plasmin, streptokinase, urokinase and elastase, steroidal and non-steroidal anti-inflammatory agents such as hydrocortisone, dexamethasone, prednisolone, methylprednisolone, promethazine, aspirin, ibuprofen, indomethacin, ketoralac, meclofenamate, tolmetin, calcium channel blockers such as diltiazem, nifedipine, verapamil, antioxidants such as ascorbic acid, carotenes and alpha-tocopherol, allopurinol, trimetazidine, antibiotics, especially noxythiolin and other antibiotics to prevent infection, prokinetic agents to promote bowel motility, agents to prevent collagen crosslinking such as cis-hydroxyproline and D-penicillamine, and agents which prevent mast cell degranulation such as disodium chromolglycate, among numerous others.

Preferred drugs for wound treatment include, but are not limited to, topical anesthetics, topical antibiotics, topical anti-fungals, topical antivitrals, and topical anti-inflammatories.

Suitable topical anesthetics include, but are not limited to, tetracaine, procaine, bupivacaine, lidocaine, lidocaine hydrochloride, benzocaine, butamben, dibucaine, pramoxine, and diphenhydramine (1% solution).

Suitable antibiotics for wound care include, but are not limited to, neosporin (Myciguent®), bacitracin (Baciguent®), combinations of the two with polymyxin B (Neosporin®, Polysporin®), metronidazole (MetroGel®), mupirocin (Bactroban®), muciprocin, erythromycin, clindamycin, tetracycline, neomycin, polymyxin B, gentamycin, azelaic acid, metronidazole, chlortetracycline, meclocycline, sulfacetamide, silver sulfadiazine, neomycin/polymyxin B sulfate/bacitracin zinc, bacitracin zinc/polymyxin B sulfate (Polysporin), and combinations thereof.

Suitable antifungals include, but are not limited to, amphotericin B, bufenafine, ciclopirox, clioquinol, clotrimazole, econazole, gentian violet, naftifine, oxiconazole, terbinafine, tolnaftate, triacetin, undecylenic acid, zinc undecylenate, and povidone iodine.

Suitable antivirals include, but are not limited to, acyclovir and penciclovir.

Suitable anti-inflammatories include, but are not limited to, aclomethasone, amcinonide, betamethasone dipropionate, betamethasone valerate, clobetasol propionate, clocortolone pivalate, desonide, desoximetasone, dexamethasone, dexamethasone sodium phosphate, diflorasone diacetate, fluocinolone acetonide, fluocinonide, flurandrenolide, fluticasone propionate, halcinonide, halobetasol propionate, hydrocortisone, hydrocortisone acetate, hydrocortisone buteprate, hydrocortisone butyrate, hydrocortisone valerate, mometasone furoate, prednicarbate, and triamcinolone acetonide.

In addition to the above agents, which generally exhibit favorable pharmacological activity related to promoting wound healing, reducing infection, other biologically or pharmaceutically active agents may be delivered by the polymers matrix fibers of the present invention to a patient in need thereof include, for example, amino acids, peptides, proteins, including enzymes, carbohydrates, antibiotics (treat a specific microbial infection), anti-cancer agents, neurotransmitters, hormones, immunological agents including antibodies, nucleic acids including antisense agents, fertility drugs, psychoactive drugs and local anesthetics, among numerous additional agents.

The invention is particularly well suited to the practice of personalized medicine, in which drug selection, dosage and delivery is tailored to an individual's genetic profile. A polymeric matrix drug-releasing matrix can be prepared to order by a formulary pharmacy in response to a physician's directions in which precise drug release profiles are constructing to address the needs of an individual patient.

The delivery of these agents will depend upon the pharmacological activity of the agent, the site of activity within the body and the physicochemical characteristics of the agent to be delivered, the therapeutic index of the agent, among other factors. One of ordinary skill in the art will be able to readily adjust the physicochemical characteristics of the present polymers and the hydrophobicity/hydrophilicity of the agent to be delivered in order to produce the intended effect. In this aspect of the invention, biologically and pharmaceutically active agents are administered in concentrations or amounts which are effective to produce an intended result. It is noted that the chemistry of polymeric composition according to the present invention can be modified to accommodate a broad range of hydrophilic and hydrophobic biologically and pharmaceutically active agents and their delivery to sites in the patient.

The present invention thus provides a single means by which a plurality of drugs may be simultaneously delivered from a single dosage form. Suitable dosage forms include subcutaneous implants, occlusive skin and buccal patches, capsules for oral or rectal administration, and the like.

The following non-limiting examples set forth hereinbelow illustrate certain aspects of the invention.

### Example 1

### Materials

Lidocaine hydrochloride (LH), mupirocin, and hexafluoroisopropanol (HFIP) were purchased from Sigma-Aldrich (St. Louis, Mo.). Poly-L-lactic acid (PLLA) Resomer L 206 was purchased from Boehringer Ingelheim Chemicals (Petersburg, Va.). Human dermal fibroblasts (HDF), CellTiter96™ AQueous Assay (MTS), were purchased from Cascade Biologics (Portland, Oreg.) and Promega Corp (Madison, Wis.) respectively. Dulbecco's Phosphate Buffered Saline, Trypsin EDTA, Gibco™ Newborn Calf Serum was purchased from Invitrogen (Carlsbad, Calif.). Staphylococcus aureus ATCC® 25923 was purchased from American Type Culture Collection (Manassas, Va.). Tryptic soy broth and agar were purchased from BD Diagnostic Systems (Sparks, Md.). Phosphate buffered saline (PBS) tablets were purchased from MP Biomedicals, CA. All the other chemicals and solvents were of analytical grade.

Electrospinning Procedures

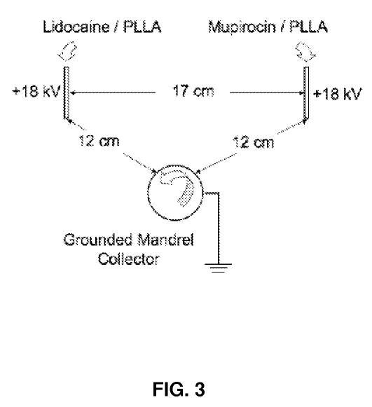

The dual spinneret electrospinning apparatus (FIG. 3) is described as follows: Polymer solutions were loaded into two programmable syringe pumps connected to two 19 gauge needles. The tip-to-collector distance was 12 cm and the distance between the two needles was 17 cm. A high voltage power supply (Gamma High Voltage Research Inc., Omaha Beach, Fla.) was used to charge the metal needle. Fibers spun from both spinnerets were simultaneously collected on a 5 cm diameter, grounded, aluminum mandrel, which was rotated at 120 rpm.

Polymer Solutions and Electrospinning Parameters

PLLA was dissolved in HFIP and gently shaken for 3 hours until the polymer was completely dissolved. A solution of LH or mupirocin in HFIP was slowly added without any visible precipitation and shaken. The homogeneous drug/polymer solution was then electrospun with the parameters listed in Table 1.

For characterization of fibers, solutions A, B, C were electrospun separately. Solutions A and B were electrospun with the dual spinneret (DS) system into a single scaffold to study release properties. Solution C was electrospun with a single spinneret (SS) apparatus for the purpose of comparison of release profiles with DS scaffold. The final scaffolds were sterilized for 14 hrs with Anprolene AN74i ethylene oxide sterilizer (Anderson Products Inc., NC) and purged for additional 4 hours followed by drying under vacuum for 36 hours.

Uniformity of Distribution

To confirm uniform spraying and mixing of the fibers in the matrix with the DS technique, Texas Red was used to stain one of the fibers. Briefly, 1% w/v of Texas Red in ethanol was suspended in a 17 wt % PLLA solution in HFIP and loaded into one syringe pump.

The other syringe pump contained a non-fluorescent solution of 17 wt % PLLA in HFIP. Conditions for electrospinning were similar to those for electrospinning of A. After drying, the fibers were viewed under a fluorescence microscope (Zeiss Axiovert 200, Thornwood, N.Y.).

Characterization of Fibers

Surface morphology of the electrospun scaffolds before and after drug release was observed on an AMRAY 1830 I scanning electron microscope (SEM). Samples for SEM were dried under vacuum, mounted on aluminum stubs, and sputter-coated with gold-palladium. Histograms of fiber diameter were generated by the measurement of approximately 160 individual fibers in 3000×SEM images using NIH-ImageJ software (http://rsb.info.nih.gov/ij/). Incorporation of drugs and polymer-drug interactions were studied by differential scanning calorimetry (DSC). The fibers were heated in DSC 2920 (TA instruments) with a heating rate of 10° C./min from −10 to 200° C. The compositions of electrospun scaffolds were quantified by Proton Nuclear Magnetic Resonance spectroscopy. Briefly, 3% w/v solutions of the DS and SS electrospun scaffolds were prepared in deuterated chloroform. Spectra were obtained with a 300 MHz Varian Mercury spectrometer (Palo Alto, Calif.). Spectrum acquisition and integration was repeated five times to assess the precision of the technique.

Drug Release

The electrospun scaffolds were placed in 5 mL of pH 7.4 phosphate buffered saline (PBS) in vertical Franz diffusion cells (Permegear Inc., Bethlehem, Pa.) with 5 replicates for each scaffold. The outer jacket of the Franz cells were maintained at 37° C. and stirred at 600 rpm and the inner compartments were covered with Parafilm®. At appropriate intervals from 1 to 72 hrs, 200 μl samples were withdrawn from the sampling port and replenished with an identical volume of fresh buffer. The drug concentrations were determined by high performance liquid chromatography (HPLC) with a Hewlett Packard 1100 system (Agilent Technologies) equipped with degasser (G1379A), autosampler (G1313A), quaternary pump (G1311A) and a UV-visible diode array (G1315A). Previously established HPLC methods were used for detection of both LH and mupirocin. In all cases, drug concentration values were corrected for the progressive dilution occurring because of the sampling pattern. Statistical analysis involved application of a two-tailed, unequal variance Student's t-test.

Antibiotic Activity

Bacterial viability tests were conducted using the rapid, modified Kirby Bauer Disc method. A 100 μl aliquot of Staphylococcus aureus reconstituted in nutrient broth and subcultured previously was spread onto an agar plate. Sections (0.5 cm diameter) of DS and SS fiber scaffolds were placed on agar plates allowing sufficient time for the drug to diffuse into the surroundings. The plate was incubated for 6 hours at 37° C., then sprayed with 0.025% MTS reagent and visualized after 10-15 min. The zones were then measured and compared against previously established interpretative criteria. Controls with no mupirocin loading were maintained separately using the same procedure.

Cell Proliferation and Morphology

Human dermal fibroblasts (500 cells/μl) were used to study cell viability on the scaffolds. Electrospun fiber scaffolds were punched (0.6 cm in diameter) and placed in sterile 96-well tissue-culture Costar® plates (Corning Incorporated, NY), 10 μl of cell suspension and 90 μl of Dulbecco's Modified Eagle Medium (DMEM) was added to each plate, and incubated for 3, 4, 6 days at 37° C. The controls contained either fibroblasts in media without a scaffold or an electrospun scaffold with media but no fibroblasts. MTS assays were performed at day 3, 4, 6 postseeding. Briefly, fresh media was added to each scaffold after aspiration of the old media and 20 μl per well of MTS solution was added. After 3 h, the supernatant was analyzed colorimetrically using a multiwell plate reader (Powerwave, Bio-Tek instruments) at 490 nm.

Scanning electron microscopy was used to examine the morphological characteristics of cells cultured onto the nanofibrous structure. Electrospun scaffolds in culture plates seeded with HDF were cultured for 3, 4 or 6 days. Loosely adherent or unbound cells were removed from the experimental wells by aspiration and the bound cells were fixed in 4% formaldehyde in a buffer (pH 7.4) for 20 min. After aspiration of the fixative and repeated washings with buffer and water, electrospun nanofibers were dehydrated in gradient ethanol solutions (50%, 70%, 85%, 95% and 100%) for 15 minutes each. After critical point drying, samples were sputtered with gold-palladium and were examined by SEM.

Characterization of Fiber Scaffolds

Fiber scaffolds containing fibers of two unique compositions were obtained using the DS electrospinning apparatus. Fluorescence microscopy of the scaffold which contained one fiber doped with Texas Red and another fiber without Texas Red showed homogenous distribution of the two fibers. In the same way, the DS electrospinning apparatus could be used to electrospin a hybrid mesh of materials of varying degradation rate, mechanical properties, or chemical functionality. Here, the technique was used to create a mesh where one fiber was loaded with an antibiotic and a second fiber was loaded with an anesthetic.

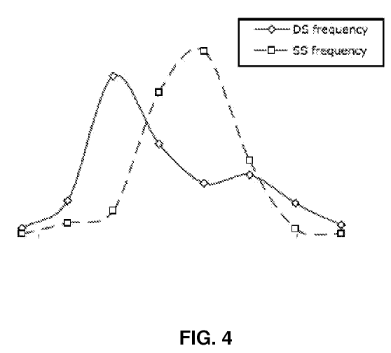

Though all solutions contained the same concentration of PLLA, the DS technique produced a scaffold with two different fiber diameter populations while the SS produced a single population of fibers with an intermediate fiber diameter, FIG. 4. This result is not surprising, since solution B had a much higher ionic strength than solution A due to the higher concentration of LH, a salt (80 wt %). Solution C, which contained 40 wt % LH, had a fiber diameter between that observed from the electrospinning of solutions A and B.

Proton NMR was used to confirm the drug-loading of the DS and SS electrospun fiber scaffolds, as a significant drip was observed from the LH solution, solution B. As expected, the LH content of the DS scaffold was lower than the amount of LH dispensed from the spinneret, Table 2. These fibers consequently had an elevated PLLA and mupirocin content. The SS scaffold, which was electrospun at 0.1 mL/hr contained the amount of drug originally added as there was no loss due to dripping.

Drug Release

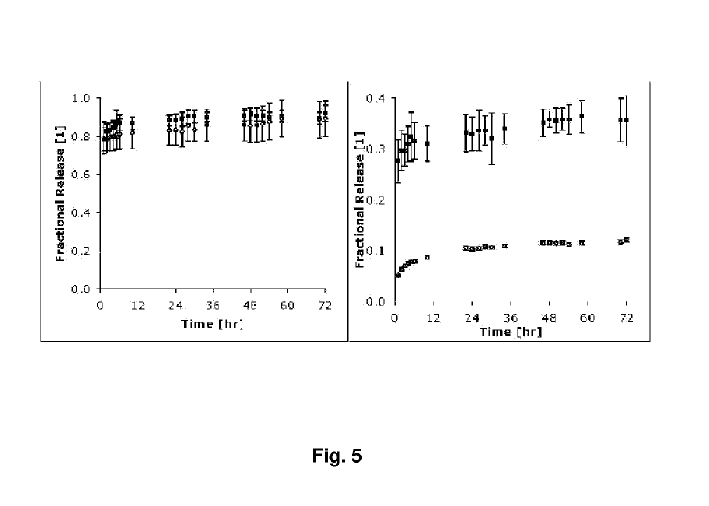

The kinetic drug release profiles are shown in FIG. 5. Both the DS and SS electrospun scaffolds eluted LH in a burst-release fashion, with 80% of the LH detected in the first hour. Over the next 71 hours, LH diffused out of the polymer matrix, achieving a cumulative release of 90%. No significant difference was found between the percent release from DS or SS fibers at 1 hr (p=0.90) and 72 hrs (p=0.63).

Though statistically indistinguishable LH release was observed in the DS and SS configurations, the SS electrospinning technique caused the undesirable burst release of 28% of the mupirocin at the first hour, while only 5% of the mupirocin diffused from the DS electrospun scaffold (p<0.001). The cumulative release at 72 hours was 12% and 36% for the DS and SS scaffolds, having nearly identical release profiles as the PLLA swelled with water and the drug diffused into the buffer. The release profiles of the four curves from 1-72 hrs were similar to that predicted by Siepmann et al. (“HPMC-matrices for controlled drug delivery: a new model combining diffusion, swelling, and dissolution mechanisms and predicting the release kinetics,” Pharm. Res., vol. 16(11), 1748-56; and “Hydrophilic matrices for controlled drug delivery: an improved mathematical model to predict the resulting drug release kinetics (the “sequential layer” model),” Pharm. Res., vol. 17(10), 1290-98 (2000)) for diffusion from a cylindrical construct. This suggests that after the initial burst release, subsequent drug content is eluted by diffusion.

Differential Scanning Calorimetry

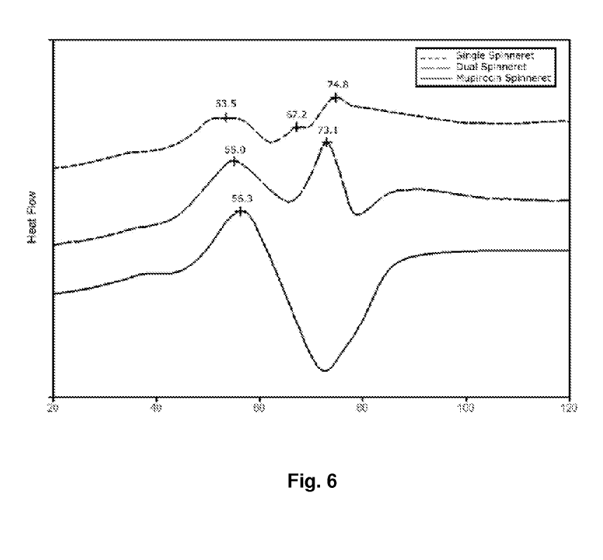

DSC of fiber scaffolds produced by the DS and SS techniques provides insight into the causation of these release profiles. FIG. 6 depicts the heat flow into fiber scaffolds as they were heated through the glass transition of the polymer and the melting points of both mupirocin (77-78° C.) and LH (74-79° C.). The electrospinning procedure causes partial alignment of the polymer chains, so after an endotherm associated with the glass transition, an exotherm due to a decrease in alignment of the PLLA chains and increase in polymer crystallinity was observed. This effect is clearly depicted in the DSC of fibers with only mupirocin, solution A, solid line. An exothermic peak for the melting of mupirocin crystals was not observed, so the mupirocin is thought to be uniformly distributed in the PLLA fiber. The DSC trace for the DS electrospinning of solutions A and B on the other hand was characterized by a large exotherm at 73° C., associated with the melting of the LH crystals. The melting point was lower than the reported range of 77-78° C., as the crystals within the PLLA matrix are not pure. Scaffolds produced by SS electrospinning of solution C had two melting points, indicating that both mupirocin and LH crystals existed within the scaffold.

The DSC data demonstrated that the DS electrospinning technique produced one population of fibers with a homogenous distribution of mupirocin throughout the PLLA matrix and a second population of fibers with crystallized LH. In contrast, when both drugs were electrospun by the traditional SS apparatus, there is a possibility that the polymer matrix did not have the capacity to hold both LH and mupirocin homogeneously within its structure, so both drugs crystallized. In drug elution, PLLA quickly absorbs water, and the crystalline drug content is released in a burst-release fashion. For this reason, a burst release of LH was observed in both the DS and SS fibers, but the undesirable burst-release of mupirocin was only observed from the SS electrospun fiber scaffold.

Crystallization of drugs in electrospun polymer fibers as a function of polymer content has been observed previously. Phase separation is considered the cause of such crystallization. Hydrochloride salts of drugs have been known to crystallize out of electrospun fibers. LH also seems to have separated out in a similar manner leading to the burst release profile.

Lipophilic drugs, on the other hand, have not been observed to crystallize out of lipophilic polymers. Mupirocin with a log P value of 3.44±0.48 (calculated by Log P DB software, ACD labs, Toronto, Canada) is a lipophilic drug and remains confined to the PLLA with no burst release even at a drug loading of 7.5 wt % in the DS fiber scaffolds. In comparison, DSC analysis of SS fibers with a relatively lower mupirocin loading of 3.75 wt % demonstrated crystallization of the drug in PLLA. This could be due to displacement from the PLLA matrix with a high LH loading. Thus, the presence of a hydrophilic salt probably enabled a burst release of a lipophilic component from a lipophilic domain.

Bacterial Susceptibility Tests

Fabrication and sterilization processes can affect the bioactivity of a compound. The modified Kirby-Bauer method was used for determining bacterial susceptibility to mupirocin eluted from ethylene oxide sterilized electrospun wound-healing scaffolds. The use of MTS reagent enabled rapid and clear delineation of the zone of inhibition. A zone of 26 mm diameter was observed for Staphylococcus aureus isolates for DS scaffold and 22 mm for SS scaffold within 6 hours. A zone diameter of 22 to 27 mm is considered acceptable for a 5 μg mupirocin disc. In our case, the DS and SS scaffolds released approximately 8 μg of mupirocin within 6 hrs, according to the release profiles and drug content from NMR results. The zone diameters obtained for these scaffolds imply that the bacterial colony is susceptible to mupirocin released from the scaffold. The zones were maintained for at least 6 days after inoculation proving that the scaffolds release significant amounts of drug throughout the course of therapy. Neither electrospinning nor ethylene oxide sterilization seem to have affected the antibiotic activity of mupirocin.

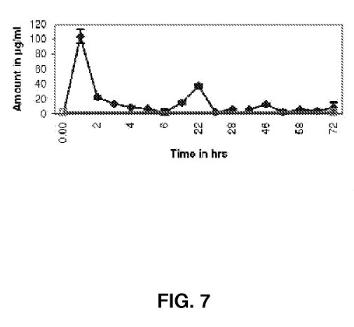

The MICs for all the strains of mupirocin-sensitive bacteria range from 0.06-0.5 μg/mL. The amount released at each time point in our DS scaffold was significantly higher than the MIC for the entire sampling period (FIG. 7). Mupirocin does not form a deposit in the skin and is metabolized into inactive monic acid. Considering that the amount of drug released by the scaffold exceeds the MIC and that mupirocin does not accumulate in the skin, it is safe to assume that the dressing will be able to maintain tissue levels of mupirocin sufficient to prevent infections in the wound for at least three days.

The slow release of mupirocin from the DS fibers ensured that the drug is released in a fashion able to maintain MIC levels satisfactorily. This prevented dose dumping at any point in the DS fiber release profile, unlike the initial hours for the SS scaffold. This is important, as excess drug can be responsible for developing antibiotic resistance and adverse events subsequent to systemic absorption. The wound dressing can be used for more than 3 days if required, for the remaining drug in the scaffold ensures continued mupirocin release and antibiotic activity. Application of commercially available ointment containing mupirocin is recommended for up to 10 days for treatment of skin lesions with a limit of 120 days on usage set by the Health and Recovery Services Administration.

Cell Viability, Attachment and Proliferation

Wound-healing scaffolds should be able to support cell proliferation and viability for fast healing of wounds. Electrospun PLLA has been seen to support growth of cells such as neural stem cells and cardiac myocytes. It is possible that inclusion of drugs may alter the cell proliferation in vivo. Lidocaine did not substantially alter wound healing or the breaking strength of the wounds. We examined the cytocompatibility of electrospun nanofibers and initial cell adhesion and spreading. The dressing was seeded with fibroblasts and calibrated MTS assays were performed to study adhesion and viability performed at day 3, 4 and 6. Human dermal fibroblasts showed a significant attachment to the scaffold at day 3 as compared to controls. The number of viable cells attached increased 3.2 times from day 3 to day 4 and 1.3 times between day 4 and day 6. The rate of cell proliferation likely decreased at day 6 because of the reduced area available for spreading and attachment. The SEM micrographs showed fibroblast attachment at each timepoint. The data implies that the drugs in the matrix do not inhibit cell proliferation and the dressing is able to support healing in addition to providing prophylactic action and pain relief.

It was determined that the dual spinneret electrospinning technique facilitated the fabrication of a polymeric wound-healing dressing with dual drug release kinetics. An anesthetic, LH, crystallized in the PLLA matrix and was eluted through a burst release mechanism for immediate relief of pain. Simultaneously, mupirocin, an antibiotic, was released through a diffusion-mediated mechanism for extended antibiotic activity. The dual spinneret electrospinning technique was able to achieve the required dual release profiles through preventing the crystallization of mupirocin within the PLLA matrix, while simultaneously allowing LH to crystallize in other PLLA fibers. The traditional single spinneret technique could not prevent the crystallization of mupirocin in the presence of 40 wt % LH. Electrospinning and ethylene oxide sterilization did not affect the antibiotic activity of mupirocin, as evidenced by the fact that the scaffold retained its antibacterial activity in vitro. We have been able to deliver the two drugs for wound healing in therapeutic concentrations for a 3-plus day therapy through a primary wound dressing. Also, if one desires to release a lipophilic drug from a lipophilic polymer, the addition of a hydrophilic salt could be used to alter the release.

### Example 2

### Methods

Poly(lactide-co-glycolide) (50:50) (PLGA) or poly(L-lactide) (PLLA) was dissolved in hexafluoroisopropanol (HFIP) and gently shaken for 3 hours till the polymer was completely dissolved. To this a solution of LH or mupirocin in HFIP was slowly added without any visible precipitation and shaken. The homogeneous drug/polymer solution was then electrospun as per the following parameters on a rotating mandrel.

| | Drug concentration | Voltage | Distance | Flow rate | Needle |
|---|---|---|---|---|---|
| Polymer % w/v | as % w/v of polymer | (kV) | (cm) | (ml/hr) | gauge |
| PLGA 20% | LH 100% | 20 | 10 | 0.5 | 19 |
| PLLA 15% | Mupirocin 25% | 15 | 18 | 0.5 | 19 |

Differential Scanning Calorimetry (DSC) was conducted on the fibers to study drug inclusion. The dried scaffolds were sectioned into uniform weight discs and placed into Franz diffusion cells (Permegear Inc., Bethlehem, Pa.) with phosphate buffered saline at 37° C. rotated at 600 rpm. Samples were withdrawn at specific times and analyzed by HPLC. An equivalent amount of fresh PBS was replaced each time.

92% of LH was released within 48 hrs with 80% burst release within the first hour. For mupirocin, an initial burst of 36 wt % being released within an hour was followed by a subsequent slow release yielding a cumulative 70 wt % release in the next 72 hours. DSC analysis demonstrates melting peaks for both drugs, indicating the presence of crystallized drug in the polymer matrix.

Variations in electrospinning parameters, polymer and solution viscosity and amount of drug loading helped achieve different release rates for both hydrophilic and hydrophobic drugs. It is possible that there exists a threshold to the amount of drug homogenously bound in a polymer matrix; beyond this amount, additional drug may form crystals in the matrix as shown by the presence of a melting point. The presence of crystallized LH and mupirocin provides therapeutic burst release, and mupirocin eluted from the polymer matrix provides sustained release to maintain significant tissue levels. These profiles will be used for simultaneous delivery of different drugs from one matrix.

The within description of the preferred embodiments should be taken as illustrating, rather than as limiting, the present invention as defined by the claims. As will be readily appreciated, numerous combinations of the features set forth above can be utilized without departing from the present invention as set forth in the claims. Such variations are not regarded as a departure from the spirit and scope of the invention, and all such modifications are intended to be included within the scope of the following claims.

What is claimed is:

1. A method of forming electrospun fiber mats from a plurality of different biodegradable polymeric fibers, in which a plurality of up to six different biodegradable polymer solutions are electrospun together by a method comprising the steps of:
providing a plurality of up to six different biodegradable polymer solutions each containing at least one biologically or pharmaceutically active material and each in communication with a needle for electrospinning a biodegradable polymer fiber from the solution; and
pumping each solution through its respective needle into an electric field under conditions effective to produce uncontrolled charged jet streams of said polymer solutions directed at a grounded rotating mandrel, thereby forming fiber threads of the biologically or pharmaceutically active compounds and polymers in the solutions that are deposited on the mandrel to form an electrospun non-woven fiber mat;
wherein said needles are positioned for co-deposition of said fiber threads from the polymer solution streams together on the mandrel to form a fiber mat.

2. The method of claim 1, wherein two or more solutions each contain a different biodegradable polymer.

3. The method of claim 1, wherein at least two solutions contain the same biodegradable polymer, but at different solution concentrations.

4. The method of claim 1, wherein at least one solution contains two or more biodegradable polymers.

5. The method of claim 1, wherein two or more solutions each contain a different biologically active or pharmaceutically active material.

6. The method of claim 1, wherein at least two solutions contain the same biologically or pharmaceutically active material, but at different solution concentrations.

7. The method of claim 1, wherein at least one solution contains two or more biologically or pharmaceutically active materials.

8. The method of claim 1, wherein at least one solution comprises an extracellular matrix protein selected from the group consisting of collagen, laminin, fibronectin, vitronectin, or a combination thereof, which is then incorporated into a fiber.

9. The method of claim 1, wherein at least one solution comprises a peptide, a cytokine, or a cell signaling molecule, or a combination thereof, which is then incorporated into a fiber.

10. The method of claim 1, wherein a first solution contains a first biodegradable polymer and a first biologically or pharmaceutically active material and a second solution contains a second biodegradable polymer and a second biologically or pharmaceutically active material.

11. The method of claim 10, wherein said first biologically or pharmaceutically active material is compatible with said first biodegradable polymer but incompatible with said second biodegradable polymer.

12. The method of claim 10, wherein said second biologically or pharmaceutically active material is compatible with said second biodegradable polymer but incompatible with said first biodegradable polymer.

13. The method of claim 10, wherein said first biologically or pharmaceutically active material is compatible with said first biodegradable polymer but incompatible with said second biodegradable polymer and said second biologically or pharmaceutically active material is compatible with said second biodegradable polymer but incompatible with said first biodegradable polymer.

14. The method of claim 1, wherein said first and second biologically or pharmaceutically active materials are incompatible with each other.

15. The method of claim 1, wherein two or more solutions contain the same biodegradable polymer and biologically or pharmaceutically active materials but different solvents.

16. The method of claim 1, wherein said biologically or pharmaceutically active material is not released from the biodegradable polymer matrix.

17. The method of claim 16, wherein said biologically or pharmaceutically active material is expressed at the fiber surface and interacts with the surrounding environment.

18. Biodegradable polymer fiber mats suitable for in vivo implantation, prepared by the electro spinning method of claim 1.

19. A medical device selected from the group consisting of barriers for the prevention of surgical adhesions, wound dressings, drug delivery devices, capsules for oral or rectal administration, subcutaneous implants, transdermal drug delivery devices, occlusive and non-occlusive skin and buccal patches, polymer scaffolds for tissue engineering, comprising the fiber mat of claim 18.

20. The medical device of claim 19, characterized by being an oral dosage comprising at least one rolled up fiber mat placed into a gelatin capsule for oral administration.
